(19) United States
(12) Patent Application Publication
(10) Pub. No.: US 2014/0005113 A1
(43) Pub. Date: Jan. 2, 2014

(54) TREATMENT OF DISORDERS WITH ALTERED VASCULAR BARRIER FUNCTION

(71) Applicant: Genentech, Inc., South San Francisco, CA (US)

(72) Inventors: Weilan Ye, Foster City, CA (US); Leon H. Parker, IV, San Francisco, CA (US); Christopher W. Wilson, San Francisco, CA (US)

(73) Assignee: Genentech, Inc., South San Francisco, CA (US)

(21) Appl. No.: 14/022,475

(22) Filed: Sep. 10, 2013

Related U.S. Application Data
(63) Continuation of application No. PCT/US2012/028588, filed on Mar. 9, 2012.
(60) Provisional application No. 61/451,540, filed on Mar. 10, 2011.

(51) Int. Cl. A61K 38/02; C12N 15/113

(52) U.S. Cl. CPC A61K 38/02; C12N 15/113
USPC 514/13.3; 514/44 A; 514/44 R

(57) ABSTRACT

The present invention provides methods of using RASIP1 agonists and antagonists to modulate vascular barrier function and regulate new vessel formation, and to treat related disorders.

### RELATED APPLICATIONS

This application is a continuation of PCT/US2012/028588, filed Mar. 9, 2012 which claims the benefit of priority to U.S. Provisional Application Ser. No. 61/451,540 filed on Mar. 10, 2011, which are incorporated by reference in their entirety.

### SEQUENCE LISTING

The instant application contains a Sequence Listing which has been submitted in ASCII format via EFS-Web and is hereby incorporated by reference in its entirety. Said ASCII copy, created on Sep. 5, 2013, is named P4616R1US_Sequence_Listing.txt and is 16,586 bytes in size.

### FIELD OF THE INVENTION

The present invention relates generally to compositions and methods that are useful for treatment of conditions and disorders associated with altered vascular barrier function. Specifically, the present invention relates to modulators of Ras-Interacting Protein 1 (Rasip1) and methods for their use.

### BACKGROUND OF THE INVENTION

In murine embryos, onset of circulation (Ji et al., Circ. Res. 92:133-35, (2003)) coincides with active growth of the major vessels such as the dorsal aorta (Walls et al., PLoS One 3:e2853, (2008); Strilic et al., Dev. Cell 17:505-15, (2009)). Subsequently, vigorous circulation ensues amid rapid vascular expansion via vasculogenesis, angiogenesis, and remodeling—dynamic processes involving extensive cell movement and positional exchange between cells (Carmeliet, Nat. Med. 6:389-95, (2000); Coultas et al., Nature 438:937-45, (2005); Jakobsson et al., Nat. Cell. Biol. 12:943-53, (2010)). These concomitant events pose a unique challenge to the developing vasculature: nascent endothelial cell-cell junctions must be stable enough to permit lumen formation, circulation and to withstand increasing shear stress, yet be flexible enough to allow cell movement during dynamic growth and remodeling. In addition, vascular permeability through regulation of cell-cell junctions are tightly regulated, as increased permeability contributes to pathologic conditions including hemorrhage, edema, ischemic stroke, inflammation, and sepsis (Dejana et al., Dev. Cell 16:209-21, (2009); Spindler et al., Cardiovascular Research 87:243-53, (2010)). Although key components in endothelial cell-cell tight and adherens junctions have been shown to critically influence vascular development and lumen stabilization (Dejana, Nat. Rev. Mol. Cell. Biol. 5:261-70, (2004); Crosby et al., Blood 105:2771-76 (2005); Dejana et al. (2009; supra)), much remains to be learned regarding the molecular ensemble that regulates endothelial cell-cell junctions in development.

A key regulator of cell-cell junction formation in both epithelial and endothelial cells is the small G protein Rap1 (Kooistra et al., J. Cell Science 120:17-22, (2007)). In many contexts, activation of protein kinase A (PKA) at the cell membrane promotes the formation of cyclic AMP (cAMP) that binds to the guanine exchange factor (GEF) Epac1 and triggers exchange of GDP to GTP on Rap1, activating the protein and triggering a cascade of molecular interactions leading to stabilization and linkage of cortical actin to proteins of adherens and tight junctional complexes (Kooistra et al., FEBS Letters 579:4966-72, (2005)). Modulation of Rap1 activity affects endothelial barrier function (Spindler et al. (2010; supra)). In this network, regulators of small G proteins, such as EPAC1, also play critical roles (Pannekoek et al., Biochim. Biophys. Acta 1788:790-96, (2009)). In the past several years, effectors of Ras and RAP1, such as MLLT4/AFADIN-6, RADIL, and KRIT1, have been identified and shown to play important roles in mediating cell-cell adhesion and migration (Boettner et al., Proc. Natl. Acad. Sci. USA 97:9064-69, (2000); Glading et al., J. Cell Biol. 161:1163-77, (2007); Mitin et al., J. Biol. Chem. 279:22353-61, (2004); Smolen et al., Genes Dev. 21:2131-36, (2007); Xu et al., Dev. Biol. 329:269-79, (2009)). In addition, Rasip1 has been shown to bind overexpressed Ras and Rap1 (Mitin et al., (2004; supra)) and knockdown of Rasip1 abolishes vessel formation in Xenopus laevis (Mitin et al., (2004; supra)); Xu et al., (2009; supra)).

Despite the many advances in our understanding of the development and maintenance of normal and pathological vasculature, there remains a need to identify targets and develop means that can supplement or enhance the efficacy of existing therapies in this area.

### SUMMARY OF THE INVENTION

The present invention is based, at least in part, on the discovery that Ras-Interacting Protein 1 (Rasip1) is essential to maintain endothelial junctional stability. Therefore, targeting Rasip1 with agents that activate it, or the signaling cascade in which it lies, is useful in the treatment of disorders with decreased vascular barrier function, including sepsis, age-related macular degeneration (AMD), edema, and hemorrhage. Accordingly, the present invention provides novel methods for treating such disorders using agents that activate Rasip1 activity. In addition, the invention is based, at least in part, on the discovery that Rasip1 is required for the formation of stable vessels. Therefore, targeting Rasip1 with agents that inhibit it, or the signaling cascade in which it lies, is useful in the treatment of disorders where new vessel formation is required, including cancers and proliferative diabetic retinopathy. Accordingly, the present invention provides novel methods for treating such disorders using agents that inhibit Rasip1 activity.

In one aspect, the invention provides a method of treating a disorder associated with altered vascular barrier function in a subject comprising administering to the subject a RASIP1 modulator. In some embodiments, the disorder is associated with reduced vascular barrier function and wherein the RASIP1 modulator is a RASIP1 agonist, including where the disorder is, e.g., sepsis, age-related macular degeneration (AMD), edema, ischemic stroke or hemorrhage. In some embodiments, the disorder is associated with increased vascular barrier function and wherein the RASIP1 modulator is a RASIP1 antagonist, including where the disorder is, e.g., hypertension.

In another aspect, the invention provides a method of reducing or inhibiting vascular barrier function in a subject in need thereof, comprising administering to the subject a RASIP1 agonist.

In another aspect, the invention provides a method of increasing or enhancing vascular barrier function in a subject in need thereof, comprising administering to the subject a RASIP1 antagonist.

In yet another aspect, the invention provides a method of treating a disorder that requires new vessel formation in a subject comprising administering to the subject a RASIP1 inhibitor, including where the disorder is, e.g., cancer or a proliferative retinopathy, including diabetic retinopathy.

In some embodiments, the RASIP1 modulator is a small molecule. In some embodiments where the RASIP1 modulator is an antagonist it is an antisense RNA, RNAi or ribozyme.

### BRIEF DESCRIPTION OF THE DRAWINGS

FIG. 1: Rasip1 knockout mice die in mid-gestation with vascular defects

(A) Brightfield image of heterozygous control (+/−) embryo at E9.0. (B) Brightfield image of Rasip1−/− embryo at E9.0, displaying smaller size, pericardial edema, and hemorrhage. (C, D) Wholemount CD31 plus CD105 immunofluorescence (red) in Rasip1+/− (C) and −/− (D) E8.5 embryos. Ventral view, rostral is to the left. (E, F) Wholemount immunofluorescence of the trunk vasculature of Rasip1+/− (E) and −/− (F) E9.0 embryos. Lateral view, rostral to the left. Red: CD31+CD105, Green: RBC autofluorescence; Blue: DAPI. Arrows: Dorsal aortae. Asterisks: Cardiac crescent. so: somite.

FIG. 2: Axial vessel defects in Rasip1 knockout mice

Transverse sections of caudal dorsal aortae from Rasip1+/− (A-D) and −/− (E-H) embryos, at 1-2 somite stage (ss) (A, E), 3-6 ss (B, F), and 7-10 ss (C, D, G, H). Note that sections in (C) and (D), as well as (G) and (H) are from adjacent sections, indicating localized collapse of the aorta in Rasip1−/− embryos at 7-10 ss. (I) Scatter plot of dorsal aortae luminal areas from Rasip1+/− (n=5) and −/− (n=5) embryos at 7-10 ss. The 20 μm² cutoff line (red) indicates functional capillary diameter. Rasip1−/− aortae display a wider variation of luminal areas. Each point represents an individual area measurement of an aorta.

FIG. 3: Disruption of rasip1 and rafadil expression in zebrafish causes aberrant EC-EC association and vascular leakage

(A) Lateral view of control morpholino oligo (ctrl) injected Tg(kdrl:EGFP)s843 zebrafish embryos at 26 hours post fertilization (hpf). Rostral is to the left. (B) rasip1 and rafadil morpholino oligos (MO) injected embryos at 26 hpf. Intersomitic vessels (ISVs) are stunted and axial vessels are morphologically abnormal. (C) Lateral views of a 27 hpf embryo showing the dorsal aorta (small bracket) and cells migrating ventrally to form the posterior cardinal vein (large bracket). (D) Lateral view of a 27 hpf double morphant. Axial positioning of angioblasts is normal (brackets), but vessel coalescence is aberrant and numerous gaps appear (arrows). (E) Fluorescent angiography of a control 54 hpf embryo, showing fluorescent microbeads (red) within vasculature (green). (F) Angiography of rasip1 and rafadil MO embryo, showing leaked extravascular microbeads.

FIG. 4: Loss of RASIP1 alters cell-cell connectivity

(A) Control (Ctrl) HUVEC angiogenic sprouting at 24 hours. (B) Sprouts formed by HUVEC stably expressing RASIP1 short hairpin RNA. Note the increase in detached cells/sprouts in RASIP1 knockdown (KD) sample. (C) Quantification of detached sprouts in Ctrl and KD samples at 24 and 48 hours. A total of 40 beads per condition from two experiments were quantified, represented as means+/−SEM. (* indicates P<0.0001, determined by unpaired, Welch-corrected t-test). (D) Migration rate (μm/min) of Ctrl and RASIP1 KD HUVEC in a two-dimensional (2D) wound healing assay. (E) Quantification of cells transiently detaching from wavefront in 2D migration assay. 21-23 movies per condition were quantified. Error bars are SEM. (F) Permeability increases in RASIP1 KD HUVEC as normalized to control. Paracellular flux was measured using 40 kDa FITC-dextran. Values are the mean of 4 independent replicate pairs. Error bars are SD. (** indicates P<0.02 determined by paired t-test).

FIG. 5: RASIP1 controls junctional refinement through GTP loading of RAP1

Ctrl (A-C) or RASIP1 KD (D-F) HUVECs were treated with EGTA followed by cBiMPS, and stained with Phalloidin (A, D; green in C, F) and VE-cadherin (B, E; red in E, F). Blue in E and F indicates nuclei (DAPI). (G) Staining of RASIP1 antibody on control HUVEC. Diffuse cytoplasmic/perinuclear as well as junctional staining (arrows) is observed. (H) Cytoplasmic and junctional signal from the RasIP1 pAb staining is markedly reduced in RASIP1 KD HUVEC. (I) GTP loading of RAP1 in control and KD HUVEC. GTP bound RAP1 is reduced in KD HUVEC as compared to control.

### DETAILED DESCRIPTION OF THE PREFERRED EMBODIMENTS

### Definitions

Unless defined otherwise, technical and scientific terms used herein have the same meaning as commonly understood by one of ordinary skill in the art to which this invention belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present invention, certain terms are defined below.

As used herein, the terms “RASIP1” or “RASIP1 polypeptide” refer to a polypeptide having the amino acid sequence of a RASIP1 polypeptide derived from nature, regardless of its mode of preparation or species. Thus, such polypeptides can have the amino acid sequence of naturally occurring RASIP1 from a human, a mouse, or any other species. A full-length human RASIP1 amino acid sequence is:

(SEQ ID NO: 1)
MLSGERKEGGSPRFGKLHLPVGLWINSPRKQLAKLGRRWPSAASVKSSSSDTGSRSSEPLPPPPPHVEL
RRVGAVKAAGGASGSRAKRISQLFRGSGTGTTGSSGAGGPGTPGGAQRWASEKKLPELAAGVAPEPPLA
TRATAPPGVLKIFGAGLASGANYKSVLATARSTARELVAEALERYGLAGSPGGGPGESSCVDAFALCDA
LGRPAAAGVGSGEWRAEHLRVLGDSERPLLVQELWRARPGWARRFELRGREEARRLEQEAFGAADSEGT
GAPSWRPQKNRSRAASGGAALASPGPGTGSGAPAGSGGKERSENLSLRRSVSELSLQGRRRRQQERRQQ
ALSMAPGAADAQIGTADPGDFDQLTQCLIQAPSNRPYFLLLQGYQDAQDFVVYVMTREQHVFGRGGNSS
GRGGSPAPYVDTFLNAPDILPRHCTVRAGPEHPAMVRPSRGAPVTHNGCLLLREAELHPGDLLGLGEHF
LFMYKDPRTGGSGPARPPWLPARPGATPPGPGWAFSCRLCGRGLQERGEALAAYLDGREPVLRFRPREE
EALLGEIVRAAAAGSGDLPPLGPATLLALCVQHSARELELGHLPRLLGRLARLIKEAVWEKIKEIGDRQ
PENHPEGVPEVPLTPEAVSVELRPLMLWMANTTELLSFVQEKVLEMEKEADQEDPQLCNDLELCDEAMA
LLDEVIMCTFQQSVYYLTKTLYSTLPALLDSNPFTAGAELPGPGAELGAMPPGLRPTLGVFQAALELTS
QCELHPDLVSQTFGYLFFFSNASLLNSLMERGQGRPFYQWSRAVQIRTNLDLVLDWLQGAGLGDIATEF
FRKLSMAVNLLCVPRTSLLKASWSSLRTDHPTLTPAQLHHLLSHYQLGPGRGPPAAWDPPPAEREAVDT
GDIFESFSSHPPLILPLGSSRLRLTGPVTDDALHRELRRLRRLLWDLEQQELPANYRHGPPVATSP.

A full-length mouse RASIP1 amino acid sequence is:

(SEQ ID NO: 2)
MLSGERKEGGSPRFGKLHLPVGLWINSPRKQLAKLGRRWPSAASVKSSSSDTGSRSSEPLPPPPPPPHV
ELRRVGAVKAAGGASGSRAKRISQLFRGSGAGGAGGPGTPGGAQRWASEKKLPELAAGVAPEPPLPTRA
AVPPGVLKIFASGLASGANYKSVLATERSTARELVAEALERYGLTGGRGAGDSGCVDAYALCDALGRPA
VGVGGGEWRAEHLRVLADAERPLLVQDLWRARPGWARRFELRGREEARRLEQEAFGAADADGTNAPSWR
TQKNRSRAASGGAALASPGPGSGSGTPTGSGGKERSENLSLRRSVSELSLQGRRRRQQERRQQALSMAP
GAADAQMVPTDPGDFDQLTQCLIQAPSNRPYFLLLQGYQDAQDFVVYVMTREQHVFGRGGPSSSRGGSP
APYVDTFLNAPDILPRHCTVRAGPEPPAMVRPSRGAPVTHNGCLLLREAELHPGDLLGLGEHFLFMYKD
PRSGGSGPARPSWLPARPGAAPPGPGWAFSCRLCGRGLQERGEALAAYLDGREPVLRFRPREEEALLGE
IVRAAASGAGDLPPLGPATLLALCVQHSARELELGHLPRLLGRLARLIKEAVWEKIKEIGDRQPENHPE
GVPEVPLTPEAVSVELRPLILWMANTTELLSFVQEKVLEMEKEADQEGLSSDPQLCNDLELCDEALALL
DEVIMCTFQQSVYYLTKTLYSTLPALLDSNPFTAGAELPGPGAELEAMPPGLRPTLGVFQAALELTSQC
ELHPDLVSQTFGYLFFFSNASLLNSLMERGQGRPFYQWSRAVQIRTNLDLVLDWLQGAGLGDIATEFFR
KLSIAVNLLCVPRTSLLKASWSSLRTDYPTLTPAQLHHLLSHYQLGPGRGPPPAWDPPPAERDAVDTGD
IFESFSSHPPLILPLGSSRLRLTGPVTDDALHRELRRLRRLLWDLEQQELPANHRHGPPVASTP.

Such RASIP1 polypeptides can be isolated from nature or can be produced by recombinant and/or synthetic means.

“Isolated” in reference to a polypeptide means that it has been purified from an natural source or has been prepared by recombinant or synthetic methods and purified. A “purified” polypeptide is substantially free of other polypeptides or peptides. “Substantially free” here means less than about 5%, preferably less than about 2%, more preferably less than about 1%, even more preferably less than about 0.5%, most preferably less than about 0.1% contamination with other source proteins.

The term “agonist” is used in the broadest sense, and includes any molecule that partially or fully activates a biological activity of a polypeptide. For example, an agonist of RASIP1 would increase the ability of RASIP1 to influence GTP loading of Rap1, or to increase cell-cell junctional stability, or to increase endothelial cell barrier function. Methods for identifying agonists of a RASIP1 polypeptide may comprise contacting the RASIP1 polypeptide with a candidate agonist molecule and measuring an appropriate detectable change in one or more biological activities normally associated with the polypeptide.

The term “antagonist” is used in the broadest sense, and includes any molecule that partially or fully blocks, inhibits, or neutralizes a biological activity of a polypeptide. For example, an antagonist of RASIP1 would partially or fully block, inhibit, or neutralize the ability of RASIP1 to modulate RAP1-GTP loading, and to regulate stable endothelial cell-cell connection. Suitable antagonist molecules specifically include antisense RNAs, ribozymes, RNAi, small organic molecules, etc. Methods for identifying antagonists of a RASIP1 polypeptide may comprise contacting the RASIP1 polypeptide with a candidate antagonist molecule and measuring a detectable change in one or more biological activities normally associated with the polypeptide.

The term “modulators” is used to refer to agonists and/or agonists collectively.

“Active” or “activity” for the purposes herein refers to form(s) of RASIP1 which retain a biological and/or an immunological activity, wherein “biological” activity refers to a biological function caused by RASIP1 other than the ability to induce the production of an antibody and an “immunological” activity refers to the ability to induce the production of an antibody against an antigenic epitope possessed by RASIP1. Principal biological activities of RASIP1 are transduction or initiation of Rap1-induced signaling and refining cellular junctions in the vasculature.

As used herein, “treatment” is an approach for obtaining beneficial or desired clinical results. For purposes of this invention, beneficial or desired clinical results include, but are not limited to, alleviation of symptoms, diminishment of extent of disease or disorder, stabilized (i.e., not worsening) state of disease, delay or slowing of disease progression, amelioration or palliation of the disease state, and remission (whether partial or total), whether detectable or undetectable. “Treatment” is an intervention performed with the intention of preventing the development or altering the pathology of a disorder. Accordingly, “treatment” may refer to therapeutic treatment or prophylactic or preventative measures. Those in need of treatment include those already with the disorder as well as those in which the disorder is to be prevented. Specifically, the treatment may directly prevent, slow down or otherwise decrease the pathology of cellular degeneration or damage, such as the pathology of tumor cells in cancer treatment, or may render the cells more susceptible to treatment by other therapeutic agents.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

A “disorder with altered vascular barrier function” is a disorder characterized by increased or decreased vascular barrier function. Disorders with increased vascular barrier function include, but are not limited to, hypertension. Disorders with decreased vascular barrier function include, but are not limited to, sepsis, age-related macular degeneration (AMD), edema, and hemorrhage.

A “disorder that requires new vessel formation” is characterized by dependency on the formation of functional new vessels. Such disorders include, but are not limited to, cancer and proliferative diabetic retinopathy.

The “pathology” of a disorder includes all phenomena that compromise the well-being of the patient.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

A “small molecule” is defined herein to have a molecular weight below about 500 Daltons.

### Methods for Carrying Out the Invention

Preparation and Identification of Antagonists of RASIP1 Activity

Screening assays for antagonist drug candidates are designed to identify compounds that bind or complex with RASIP1 polypeptides, or otherwise interfere with their activity and/or interaction with other cellular proteins.

Small molecules may have the ability to act as RASIP1 agonists or antagonists and thus to be therapeutically useful. Such small molecules may include naturally occurring small molecules, synthetic organic or inorganic compounds and peptides. However, small molecules in the present invention are not limited to these forms. Extensive libraries of small molecules are commercially available and a wide variety of assays are taught herein or are well known in the art to screen these molecules for the desired activity.

In some embodiments, small molecule RASIP1 agonists or antagonists are identified by their ability to activate or inhibit one or more of the biological activities of RASIP1. Thus a candidate compound is contacted with RASIP1 and a biological activity of RASIP1 is then assessed. In one embodiment the ability of RASIP1 to modulate RAP1 GTP loading is assessed. A compound is identified as an agonist where the biological activity of RASIP1 is stimulated and a compound is identified as an antagonist where the biological activity of RASIP1 is inhibited.

Another potential RASIP1 antagonist is an antisense RNA or DNA construct prepared using antisense technology, where, e.g., an antisense RNA or DNA molecule acts to block directly the translation of mRNA by hybridizing to targeted mRNA and preventing protein translation. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature RASIP1 polypeptides herein, is used to design an antisense RNA oligonucleotide of from about 10 to 40 base pairs in length. A DNA oligonucleotide is designed to be complementary to a region of the gene involved in transcription (triple helix—see, Lee et al., Nucl. Acids Res. 6:3073 (1979); Cooney et al., Science 241:456 (1988); Dervan et al., Science 251:1360 (1991)), thereby preventing transcription and the production of RASIP1. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex helix formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex. The antisense RNA oligonucleotide hybridizes to the mRNA in vivo and blocks translation of the mRNA molecule into RASIP1 (antisense—Okano, Neurochem. 56:560 (1991); Oligodeoxynucleotides as Antisense Inhibitors of Gene Expression (CRC Press: Boca Raton, Fla., 1988).

The antisense oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger, et al., Proc. Natl. Acad. Sci. U.S.A. 86:6553-6556 (1989); Lemaitre, et al., Proc. Natl. Acad. Sci. U.S.A. 84:648-652 (1987); PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., BioTechniques 6:958-976 (1988)) or intercalating agents (see, e.g., Zon, Pharm. Res. 5:539-549 (1988)). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an anomeric oligonucleotide. An anomeric oligonucleotide forms specific double-stranded hybrids with complementary RNA in which, contrary to the usual units, the strands run parallel to each other (Gautier, et al., Nucl. Acids Res. 15:6625-6641 (1987)). The oligonucleotide is a 2′-0-methylribonucleotide (Inoue, et al., Nucl. Acids Res. 15:6131-6148 (1987)), or a chimeric RNA-DNA analogue (Inoue, et al., FEBS Lett. 215:327-330 (1987)).

In some embodiments, the antagonists are inhibitory duplex RNAs, e.g. siRNA, shRNA, etc.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein, et al. (Nucl. Acids Res. 16:3209 (1988)), methylphosphonate oligonucleotides can be prepared by use of controlled pore glass polymer supports (Sarin, et al., Proc. Natl. Acad. Sci. U.S.A. 85:7448-7451 (1988)), etc.

The oligonucleotides described above can also be delivered to cells such that the antisense RNA or DNA may be expressed in vivo to inhibit production of RASIP1. When antisense DNA is used, oligodeoxyribonucleotides derived from the translation-initiation site, e.g., between about −10 and +10 positions of the target gene nucleotide sequence, are preferred.

Potential antagonists further include small molecules that bind to RASIP1, thereby blocking its activity. Examples of small molecules include, but are not limited to, small peptides or peptide-like molecules, preferably soluble peptides, and synthetic non-peptidyl organic or inorganic compounds.

Additional potential antagonists are ribozymes, which are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology 4:469-471 (1994), and PCT publication No. WO 97/33551 (published Sep. 18, 1997).

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions which form complementary base pairs with the target mRNA. The sole requirement is that the target mRNA have the following sequence of two bases: 5′-UG-3′. The construction and production of hammerhead ribozymes is well known in the art and is described more fully in Myers, Molecular Biology and Biotechnology: A Comprehensive Desk Reference, VCH Publishers, New York (1995), (see especially FIG. 4, page 833) and in Haseloff and Gerlach, Nature, 334:585-591 (1988), which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., Science, 224:574-578 (1984); Zaug and Cech, Science, 231:470-475 (1986); Zaug, et al., Nature, 324:429-433 (1986); published International patent application No. WO 88/04300 by University Patents Inc.; Been and Cech, Cell, 47:207-216 (1986)). The Cech-type ribozymes have an eight base pair active site that hybridizes to a target RNA sequence whereafter cleavage of the target RNA takes place. The invention encompasses those Cech-type ribozymes that target eight base-pair active site sequences that are present in the target gene.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes, unlike antisense molecules, are catalytic, a lower intracellular concentration is required for efficiency.

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

### Administration Protocols, Schedules, Doses, and Formulations

The RASIP1 agonists and antagonists are pharmaceutically useful as a prophylactic and therapeutic agent for various disorders and diseases as set forth above.

Therapeutic compositions of the agonists or antagonists are prepared for storage by mixing the desired molecule having the appropriate degree of purity with optional pharmaceutically acceptable carriers, excipients, or stabilizers (Remington's Pharmaceutical Sciences, 16th edition, Osol, A. ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. Carriers for topical or gel-based forms of antagonist include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations. RASIP1 antagonists will typically be formulated in such vehicles at a concentration of about 0.1 mg/ml to 100 mg/ml.

Another formulation comprises incorporating RASIP1 agonists or antagonists into formed articles. Such articles can be used in modulating endothelial cell growth and angiogenesis. In addition, tumor invasion and metastasis may be modulated with these articles.

RASIP1 agonists or antagonists to be used for in vivo administration must be sterile. This is readily accomplished by filtration through sterile filtration membranes, prior to or following lyophilization and reconstitution. If in lyophilized form, RASIP1 agonists or antagonists is typically formulated in combination with other ingredients for reconstitution with an appropriate diluent at the time for use. An example of a liquid formulation of RASIP1 agonists or antagonists is a sterile, clear, colorless unpreserved solution filled in a single-dose vial for subcutaneous injection. Preserved pharmaceutical compositions suitable for repeated use may contain, for example, depending mainly on the indication and type of polypeptide:

RASIP1 agonist or antagonist;

a buffer capable of maintaining the pH in a range of maximum stability of the polypeptide or other molecule in solution, preferably about 4-8;

a detergent/surfactant primarily to stabilize the polypeptide or molecule against agitation-induced aggregation;

an isotonifier;

a preservative selected from the group of phenol, benzyl alcohol and a benzethonium halide, e.g., chloride; and

water.

If the detergent employed is non-ionic, it may, for example, be polysorbates (e.g., POLYSORBATE™ (TWEEN™) 20, 80, etc.) or poloxamers (e.g., POLOXAMER™ 188). The use of non-ionic surfactants permits the formulation to be exposed to shear surface stresses without causing denaturation of the polypeptide. Further, such surfactant-containing formulations may be employed in aerosol devices such as those used in a pulmonary dosing, and needleless jet injector guns (see, e.g., EP 257,956).

An isotonifier may be present to ensure isotonicity of a liquid composition of RASIP1 agonists or antagonists, and includes polyhydric sugar alcohols, preferably trihydric or higher sugar alcohols, such as glycerin, erythritol, arabitol, xylitol, sorbitol, and mannitol. These sugar alcohols can be used alone or in combination. Alternatively, sodium chloride or other appropriate inorganic salts may be used to render the solutions isotonic.

The buffer may, for example, be an acetate, citrate, succinate, or phosphate buffer depending on the pH desired. The pH of one type of liquid formulation of this invention is buffered in the range of about 4 to 8, preferably about physiological pH.

The preservatives phenol, benzyl alcohol and benzethonium halides, e.g., chloride, are known antimicrobial agents that may be employed.

Therapeutic polypeptide compositions described herein generally are placed into a container having a sterile access port, for example, an intravenous solution bag or vial having a stopper pierceable by a hypodermic injection needle. The formulations may be administered as repeated intravenous (i.v.), subcutaneous (s.c.), or intramuscular (i.m.) injections, or as aerosol formulations suitable for intranasal or intrapulmonary delivery (for intrapulmonary delivery see, e.g., EP 257,956). The formulations are preferably administered as intravitreal (IVT) or subconjuctival delivery.

Therapeutic polypeptides can also be administered in the form of sustained-released preparations. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res. 15:167-277 (1981) and Langer, Chem. Tech. 12:98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers 22:547-556 (1983)), non-degradable ethylene-vinyl acetate (Langer et al., supra), degradable lactic acid-glycolic acid copolymers such as the Lupron Depot® (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Sustained-release RASIP1 agonists or antagonists compositions also include liposomally entrapped antagonists. Such liposomes are prepared by methods known per se: DE 3,218,121; Epstein et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324. Ordinarily the liposomes are of the small (about 200-800 Angstroms) unilamellar type in which the lipid content is greater than about 30 mol. % cholesterol, the selected proportion being adjusted for the optimal therapy.

The therapeutically effective dose of a RASIP1 agonist or antagonist will, of course, vary depending on such factors as the pathological condition to be treated (including prevention), the method of administration, the type of compound being used for treatment, any co-therapy involved, the patient's age, weight, general medical condition, medical history, etc., and its determination is well within the skill of a practicing physician. Accordingly, it will be necessary for the therapist to titer the dosage and modify the route of administration as required to obtain the maximal therapeutic effect.

With the above guidelines, the effective dose generally is within the range of from about 0.001 to about 1.0 mg/kg, more preferably about 0.01-1.0 mg/kg, most preferably about 0.01-0.1 mg/kg.

The route of agonist or antagonist administration is in accord with known methods, e.g., by injection or infusion by intravenous, intramuscular, intracerebral, intraperitoneal, intracerobrospinal, subcutaneous, intraocular (including intravitreal), intraarticular, intrasynovial, intrathecal, oral, topical, or inhalation routes, or by sustained-release systems as noted.

Examples of pharmacologically acceptable salts of molecules that form salts and are useful hereunder include alkali metal salts (e.g., sodium salt, potassium salt), alkaline earth metal salts (e.g., calcium salt, magnesium salt), ammonium salts, organic base salts (e.g., pyridine salt, triethylamine salt), inorganic acid salts (e.g., hydrochloride, sulfate, nitrate), and salts of organic acid (e.g., acetate, oxalate, p-toluenesulfonate).

The following Examples are offered for illustrative purposes only, and are not intended to limit the scope of the present invention in any way.

The disclosures of all patent and literature references cited in the present specification are hereby incorporated by reference in their entirety.

### EXAMPLES

Commercially available reagents referred to in the Examples were used according to manufacturer's instructions unless otherwise indicated. All references cited herein are hereby incorporated by reference.

### Example 1

### Rasip1−/− Mice Exhibit Abnormal Cardiovascular Development

In a bioinformatics screen for genes whose expression is enriched in the vasculature, we identified RASIP1 as being highly expressed in endothelial cells (EC), and we confirmed vascular selective expression by in situ hybridization in mouse embryos. To investigate the in vivo role of Rasip1, we generated a conventional knockout targeting exon 3 of the mouse Rasip1 locus, predicted to create a truncated protein of approximately 40 amino acids. Briefly, a BAC-based targeted vector was designed with loxP sites flanking exon 3 of the mouse Rasip1 locus. This construct was introduced into C57BL/6 ES cells and recombination events screened by PCR and sequencing. To generate a conventional knockout, the targeted ES cells were infected with adenovirus encoding Cre recombinase to delete exon 3. Two founder lines backcrossed and maintained on a pure C57BL/6 background were selected for analysis and yielded identical phenotypes. Genotyping was performed by PCR using the RED Extract-N-Amp kit (Sigma).

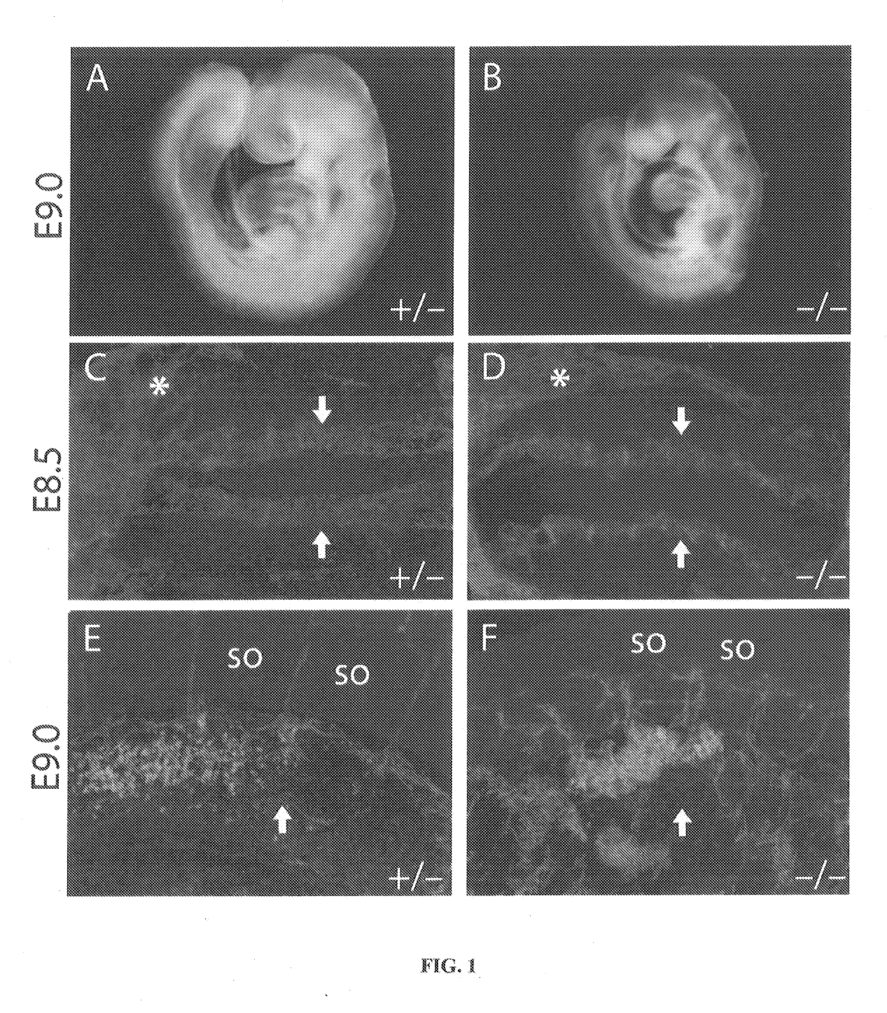

No homozygous mutant offspring were obtained from heterozygous parents, so we examined Rasip1 mutant embryos. In contrast to wildtype and heterozygous littermates at embryonic day (E) 9.0, Rasip1−/− animals were slightly smaller in size, pale, and displayed multifocal hemorrhage and pericardial edema, indicative of defects in the cardiovascular system (FIG. 1A, B). Yolk sacs of Rasip1−/− embryos were pale and exhibited abnormal vascular morphology (data not shown). At E10.5, Rasip1−/− mutant embryos were markedly smaller than control littermates with exacerbated edema and hemorrhage. Rasip1−/− embryos were not detected past E12.5, and no overt morphological defects were seen at stages earlier than E8.75. We confirmed loss of full-length RASIP1 protein in null embryos at E9.0 using a rabbit polyclonal antibody directed against the extreme C-terminus of RASIP1. No full-length protein corresponding to the predicted molecular weight was observed in Rasip1−/− whole embryo lysates. Taken together, these data demonstrated that targeted disruption of mouse Rasip1 results in abnormal cardiovascular development and mid-gestational lethality.

We next analyzed the vasculature in Rasip1 knockout mice in more detail. Whole mount embryos at 7-10 somite stage (ss) stained with EC markers revealed that the paired dorsal aortae (DA) of Rasip1−/− embryos were assembled in the appropriate lateral positions (FIG. 1C, 1D). However, the width of the DA was irregular along the rostral-caudal axis with the appearance of poorly formed or refined cell-cell contacts. By 18 ss, the cardinal veins (CV) and DA appeared to either collapse or dilate in Rasip1−/− embryos, with accompanying hemorrhage. ECs were also disorganized or appear dispersed (FIG. 1E,F). Red blood cells (RBC) appeared to collect within the remnants of vessels, or were found in extravascular space.

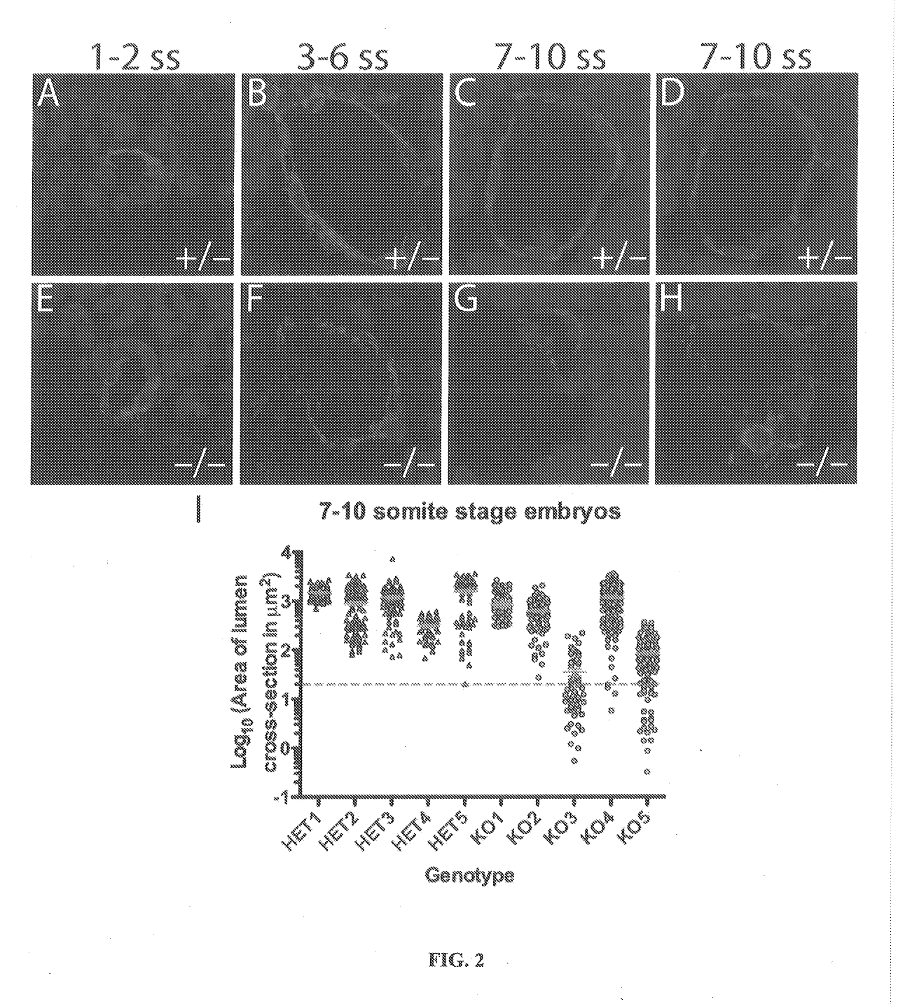

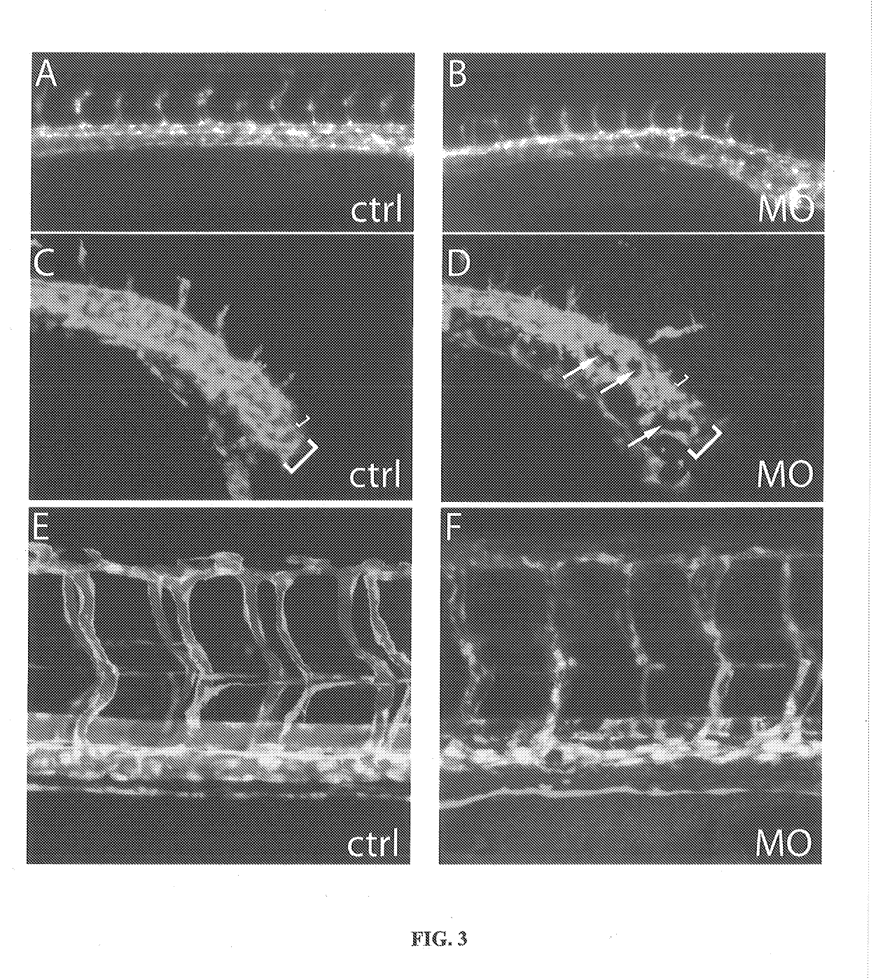

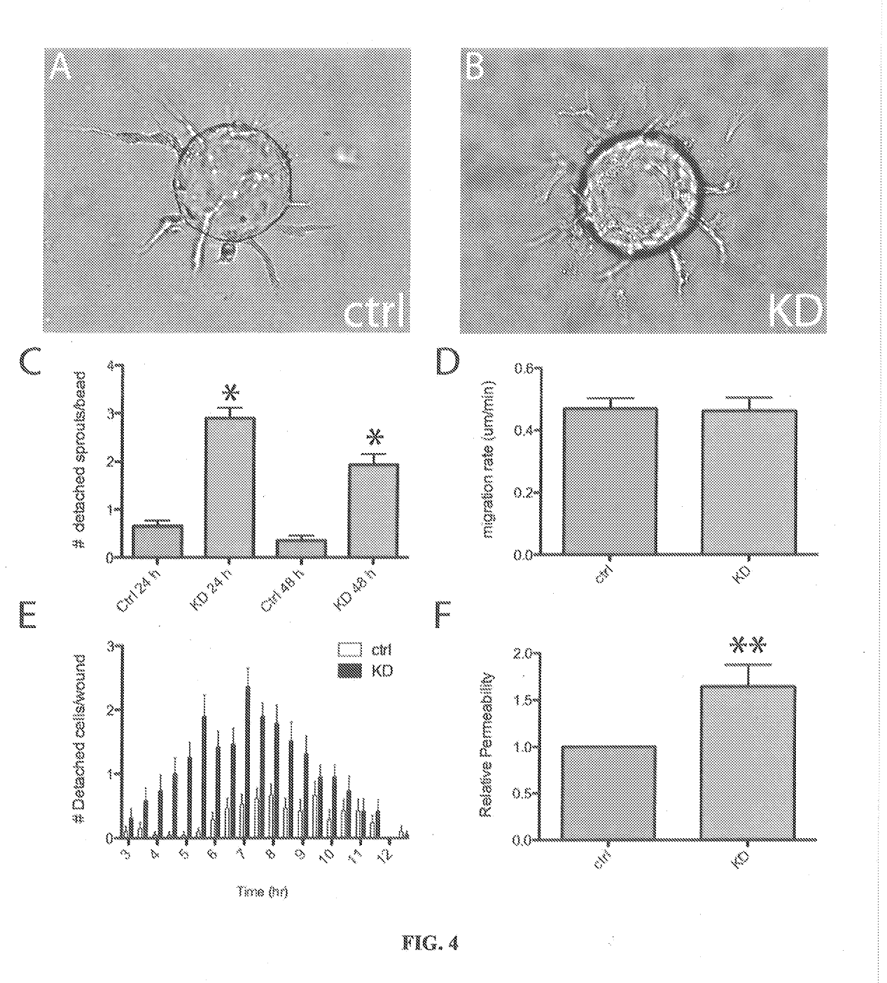

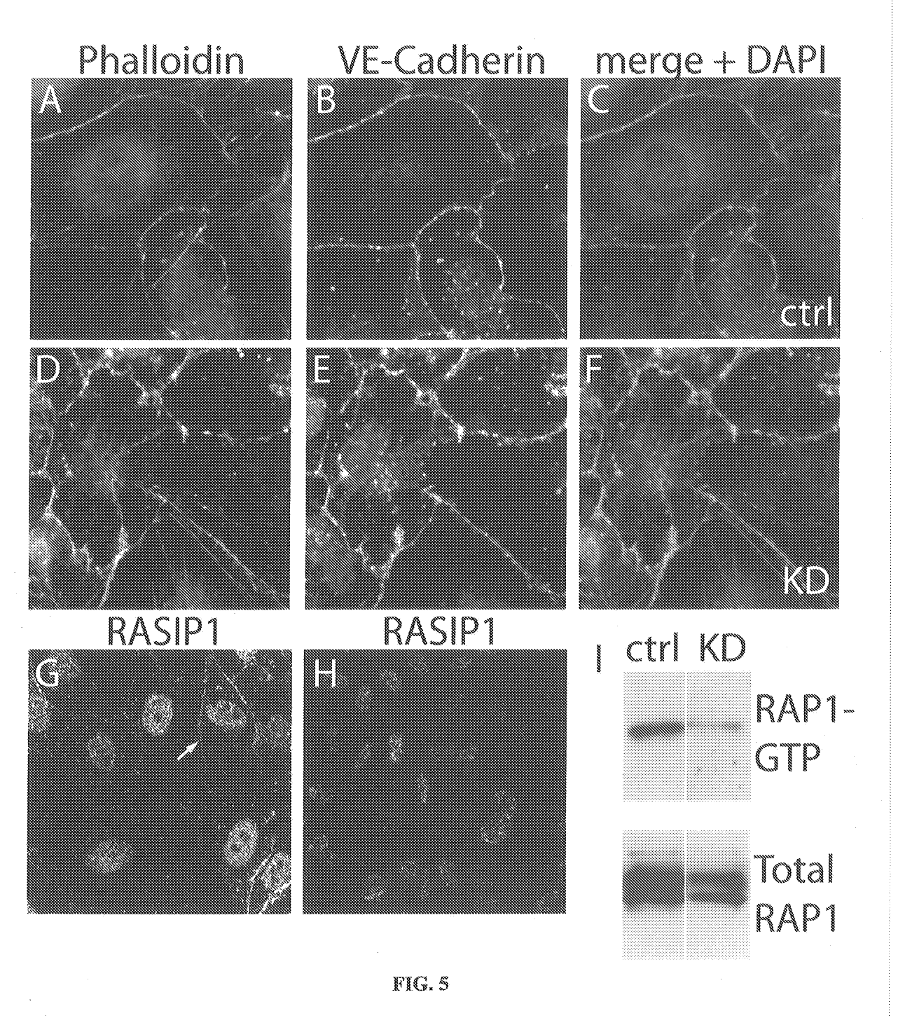

Formation of the murine DA initiates when clusters of ECs elongate into cords, accompanied by extracellular lumen formation, defined as a space larger than 5 μm between ECs (Strilic et al., Dev. Cell 17:505-15 (2009)). This process is largely complete by 6 ss, although the diameter of the DA continues to enlarge, and angiogenic sprouting off the vessels occurs (Strilic et al., supra (2009)). To rigorously determine whether vascular lumen formed in Rasip1−/− embryos, we analyzed transverse sections of DA from mutant and littermate control embryos at 1-2 ss, 3-6 ss, and 7-10 ss. At 1-2 ss, small spaces (slits) were detected between EC clusters in both Rasip1+/− and −/− embryos (FIG. 2), which generally had a lumenal cross-sectional area around 20 μm². By 3-6 ss, DA had developed a luminal space greater than 20 μm² regardless of genotype, although the extent of this space was considerably more variable in Rasip1−/− embryos (FIG. 2, data not shown). From 7-10 ss, Rasip1−/− embryos showed extensive variation in the size of the DA luminal space along the rostral-caudal axis, even between adjacent sections that are 20 μm apart, with pronounced indications of vascular collapse in one section adjacent to another with seemingly normal lumen (FIG. 2G,H). This phenomenon was also observed when examining contralaterally paired DA, and persisted through later stages of embryogenesis. We conclude that loss of Rasip1 does not preclude initial establishment of vascular lumen, but leads to a slight delay in lumenal expansion, followed by localized dilation or collapse of the major axial vessels. Further, the mutant vasculature appears to be partially functional, allowing circulation of primitive erythrocytes for a period prior to the onset of hemorrhage.

### Example 2

### Investigating the Role of Rasip1 in Zebrafish

We next used zebrafish to examine the role of Rasip1 in vascular development at the cellular level. Using the human RASIP1 Ras-associated and Forkhead-associated domains as the query sequence, we identified two expressed sequence tag (EST) clones in the Ensembl database (www.ensembl.org) as potential RASIP1 orthologs. EST clones with partial sequences of rasip1 and rafadil (GenBank: BM03633, EB781618.1) were from Open Biosystems. Additional cDNAs were cloned by 5′ and 3′ RACE with the SMART RACE cDNA Amplification kit (Clontech) using KOD Hot Start DNA polymerase (EMD Biosciences). Sequences of RACE clones were used to obtain full-length cDNAs by RT-PCR using total RNA from 30 hours post-fertilization zebrafish embryos. rasip1 and rafadil cDNAs were subcloned using TopoXL PCR cloning kit (Invitrogen) into pCS2+ for in vitro synthesis of 5′ capped mRNA using the Message Machine Sp6 kit (Ambion). The ESTs were fully sequenced and used to clone both full-length cDNAs. The first encodes a protein with high sequence similarity to mouse and human Rasip1/RASIP1, which we infer to be the zebrafish ortholog. The second gene bore similarity to both zebrafish rasip1 and RADIL, a related member of the afadin-6 family (Smolen et al., Genes Dev. 21:2131-36 (2007)). We named this gene rafadil (for Ras-Associated, Forkhead-Associated, DILute domain protein). Phylogenetic analysis indicates that rafadil is a fish-specific gene, which likely arose through an ancestral gene duplication event. Both rasip1 and rafadil are highly expressed in the developing vasculature.

In zebrafish, development and lumenization of the major axial vessels is independent of circulation (Isogai et al. 2003), and we sought to visualize vascular development using the established Tg(kdrl:EGFP)^{s843} line (Beis et al., Development 132:4193-204 (2005)). Knockdown of the zebrafish rasip1 via morpholino oligonucleotide injection into Tg(kdrl:EGFP)^{s843} embryos resulted in the formation of aberrant and leaky intersomitic vessels (ISVs), whereas knockdown of rafadil had no overt effect. Combined knockdown of both rasip1 and rafadil resulted in notable morphological alteration in the axial vessels and stunted ISVs (FIG. 3A, B). In a staged developmental series, we observed normal formation of angioblast aggregates ventral to the notochord in control and double morphant embryos at 22 ss, as previously reported (Parker et al., Nature 428:754-58 (2004); Jin et al., Development 132:5199-209 (2005)). At 24 ss, a subset of angioblasts dissociate from the midline aggregates and migrate/sprout ventrally (Herbert et al., Science 326:294-98 (2009)), which subsequently coalesce into the PCV around 26 ss. Although angioblasts in the morphants dissociated from the midline aggregates and migrated properly, they were defective in coalesceing into the PCV, as gaps indicating aberrant cell-cell connections appeared and persisted (FIG. 3C, D). These defects ultimately led to a dysfunctional, leaky vasculature, as assessed by micro-angiography (FIG. 3E, F). Vascular defects were rescued with injection of in vitro transcribed RNA encoding rasip1 and rafadil.

Taken together, our data in the zebrafish indicate that loss of rasip1/rafadil expression leads to formation of unstable vasculature that leaks and collapses as a result of compromised EC-EC coherence.

### Example 3

### Investigating the Role of Rasip1 in Cultured Human Cells

To better understand the cellular changes underlying the vascular defects in Rasip1 mutant embryos we undertook a series of in vitro analyses of human umbilical vein endothelial cells (HUVEC) lacking significant RASIP1 expression. Knockdown of RASIP1 protein with both siRNA and lentivirally delivered shRNA was confirmed by qPCR and Western blot, and had no significant effect on the ability of HUVEC to proliferate, migrate, or survive under stress. We perfomed a three-dimensional angiogenic sprouting assays as described (Nakatsu et al., Microvasc. Res. 66:102-12 (2003)). DIC images of sprouts were acquired on a Zeiss Observer Z.1, with a 10× Fluar objective, NA 0.5, using Slidebook software (Intelligent Imaging Innovations). In the three-dimensional angiogenic sprouting assay we observed loss of RASIP1 resulted in fragmentation of sprouts (FIG. 4A,B), suggesting deficiencies in maintaining connections between cells. A “detachment ratio”, measuring the number of fragmented sprouts in relation to the total number of sprouts, showed a significant increase in RASIP1 knockdown HUVEC versus control (FIG. 4C). At later time points (3-5 days), where control sprouts have established lumen, RASIP1 knockdown sprouts showed transient lumen formation, with an increased number of break points over control HUVEC. Thus, as in mice and fish, our in vitro angiogenesis system provides strong evidence of disrupted cell-cell contacts and transient and unstable lumen formation resulting from loss of RASIP1 expression.

We set out to determine which of the following reasons account for the lack of cell-cell cohesion in RASIP1 knockdown HUVEC: a change in migratory capacity of the cells, alteration in adhesion to extracellular matrix (ECM), or a change in cell-cell junction composition or stability. In contrast to a prior report, which utilized transformed MS 1 cells (Xu et al., Dev. Biol. 329:269-79 (2009)), we observed no significant difference in the ability of control and RASIP1 knockdown HUVEC to migrate in a scratch wound assay where confluent HUVEC monolayers in 24-well plates were scratched with pipette tips and monitored for 24 hours in EGM-2 (Lonza) using an Essen Incucyte system (Essen BioScience) (FIG. 4D). However, an increase in the number of cells that detached briefly and reassembled with the migrating wavefront was seen (FIG. 4E). We did not detect a significant difference in the ability of control and knockdown HUVEC to adhere either to type I collagen or fibronectin (Parker et al., Nature 428:754-58 (2004)). We then used a paracellular flux assay to measure junctional integrity (Zhao et al., J. Cell. Biol. 189:955-65 (2010)), and found that passage of 40 kDa FITC-dextran increased by approximately 50-60% in RASIP1 knockdown HUVEC compared to control (FIG. 4F). Taken together, our data indicate that loss of RASIP1 does not significantly impact the ability of ECs to migrate or adhere to common ECM substrates, but instead impairs cell-cell connectivity

Next, we investigated whether knockdown of RASIP1 impacted the ability of tight or adherens junctions to form. In steady-state, confluent, serum-starved HUVEC cultures, loss of RASIP1 did not alter the localization or protein levels of tight junction (CLAUDIN-5, OCCLUDIN, ZO-1), adherens junction (α-CATENIN, β-CATENIN, p120-CATENIN, VE-cadherinCADHERIN), focal adhesion (activated β1-INTEGRIN, FAK, PAXILIN, vinculinVINCULIN) or actin cytoskeleton-related proteins (alpha-ACTININ, non-muscle myosin IIA) in discontinuous junctions formed under this culture condition (Millan et al., BMC Biology 8:11 (2010)). To monitor the initiation and formation of new, continuous EC-EC junctions, we used EGTA to disrupt calcium-dependent adherens junctions (Sakurai et al., Molec. Biol. Cell. 17:966-76 (2006)) followed by treatment with Sp-5,6-diCl-cBiMPS, an EPACl-selective cAMP analog expected to activate the small GTPase RAP1, and thus promote junctional re-assembly (Christensen et al., J. Biol. Chem. 278:35394-402 (2003)). Under these conditions, junctions re-assembled into tight complexes 30-60 minutes after exposure to cBiMPS, as determined by VE-CADHERIN, β-CATENIN, CLAUDIN-5 and ZO-1 staining (FIG. 5C), with accompanying association of a thin belt of cortical actin that closely paralleled the junctional markers (FIG. 5A). Remarkably, staining of cortical ACTIN, VE-CADHERIN, β-CATENIN and ZO-1 was either irregular and/or discontinuous in RASIP1 knockdown HUVEC in this assay (FIG. 5D-F). Numerous ‘spikes’ or short actin filaments emerged perpendicular to cell-cell contact, and VE-cadherin and phalloidin staining was irregular and jagged in the RASIP1 knockdown cells as opposed to the refined, compact junctions formed in control cells (FIG. 5A-F). The lack of refinement of both tight and adherens junction markers, as well as ACTIN, suggests that loss of RASIP1 affects a process that coordinates the linkage of sub-membranous actin to junctional proteins.

The inability of Rasip1 knockdown cells to form continuous, refined junctions in a model requiring RAP1 stimulation of barrier formation prompted us to investigate a direct relationship between RASIP1, junctions, and RAP1. First, we examined RASIP1 localization using our RASIP1 antibody. In the barrier reformation model, RASIP1 signal was prominent at newly formed cell-cell junctions and overlapped with β-CATENIN staining, indicating junctional or sub-membranous localization (FIG. 5G, data not shown). This signal was not seen in RASIP1 knockdown HUVEC (FIG. 5H), confirming antibody specificity. We then reinforced a functional link to EPAC1/RAP1 by confirming our results with cBiMPS using the Epac1-specific cAMP analog 8-pCPT-2′-O-Me-cAMP. Finally, given the reported association between RASIP1 and RAP1A (Mitin et al., J. Biol. Chem. 279:22353-61, (2004)), we investigated whether GTP loading of RAPT was compromised in RASIP1 knockdown HUVEC. Significant levels of RAP1-GTP is seen in HUVEC treated with cBiMPS, this level is markedly decreased in RASIP1 knockdown HUVEC. Thus, loss of RASIP1 affects GTP loading of RAP1, which may explain the lack of refinement of nascent junctions. As RASIP1 does not possess GAP or GEF domains, we speculate that it may affect RAP1 function by controlling its localization, or accessibility of factors such as EPAC1. We propose that the unrefined junctions observed in the barrier reformation assay are a hallmark of the increased fragility of EC-EC junctions that result when the cells are exposed to contractile or tensile forces. This underlying defect in junctional stability explains the inability of Rasip1 mouse mutants and fish morphants to form stable lumen, as affected nascent vessels are unlikely to constantly withstand increased tensional forces brought about by vascular expansion, as well as to resist the hydrodynamic forces of circulation.

Our work demonstrates an essential role for Rasip1 in vertebrate vascular development, and shows that Rasip1 is critical for stabilizing new cell-cell junctions during active vascular growth. These studies lay the groundwork for the investigation of the role of Rasip1 in pathologic conditions affected by compromised vascular junctional integrity, and we anticipate that activation of RASIP1, or the signaling cascade in which it lies, would have protective effects in diseases with altered vascular barrier function, such as sepsis, age-related macular degeneration (AMD), edema, and hemorrhage.

The foregoing written specification is considered to be sufficient to enable one skilled in the art to practice the invention. However, various modifications of the invention in addition to those shown and described herein will be apparent to those skilled in the art from the foregoing description and fall within the scope of the appended claims.

What is claimed is:

1. A method of treating a disorder associated with altered vascular barrier function in a subject comprising administering to the subject a RASIP1 modulator.

2. The method of claim 1, wherein the disorder is associated with reduced vascular barrier function and wherein the RASIP1 modulator is a RASIP1 agonist.

3. The method of claim 2, wherein the disorder is selected from the group consisting of sepsis, age-related macular degeneration (AMD), edema, ischemic stroke and hemorrhage.

4. The method of claim 1, wherein the disorder is associated with increased vascular barrier function and wherein the RASIP1 modulator is a RASIP1 antagonist.

5. The method of claim 4, wherein the disorder is hypertension.

6. The method of any of claims 1-5, wherein the RASIP1 modulator is a small molecule.

7. A method of reducing or inhibiting vascular barrier function in a subject in need thereof, comprising administering to the subject a RASIP1 agonist.

8. A method of increasing or enhancing vascular barrier function in a subject in need thereof, comprising administering to the subject a RASIP1 antagonist.

9. A method of treating a disorder that requires new vessel formation in a subject comprising administering to the subject a RASIP 1 inhibitor.

10. The method of claim 9, wherein the disorder is cancer or a proliferative retinopathy.


SEQUENCE LISTING (2 sequences)

SEQ ID NO: 1 - protein, 963 aa, Homo sapiens
MLSGERKEGGSPRFGKLHLPVGLWINSPRKQLAKLGRRWPSAASVKSSSSDTGSRSSEPLPPPPPHVELRRVGAVKAAGGASGSRAKRISQLFRGSGTGTTGSSGAGGPGTPGGAQRWASEKKLPELAAGVAPEPPLATRATAPPGVLKIFGAGLASGANYKSVLATARSTARELVAEALERYGLAGSPGGGPGESSCVDAFALCDALGRPAAAGVGSGEWRAEHLRVLGDSERPLLVQELWRARPGWARRFELRGREEARRLEQEAFGAADSEGTGAPSWRPQKNRSRAASGGAALASPGPGTGSGAPAGSGGKERSENLSLRRSVSELSLQGRRRRQQERRQQALSMAPGAADAQIGTADPGDFDQLTQCLIQAPSNRPYFLLLQGYQDAQDFVVYVMTREQHVFGRGGNSSGRGGSPAPYVDTFLNAPDILPRHCTVRAGPEHPAMVRPSRGAPVTHNGCLLLREAELHPGDLLGLGEHFLFMYKDPRTGGSGPARPPWLPARPGATPPGPGWAFSCRLCGRGLQERGEALAAYLDGREPVLRFRPREEEALLGEIVRAAAAGSGDLPPLGPATLLALCVQHSARELELGHLPRLLGRLARLIKEAVWEKIKEIGDRQPENHPEGVPEVPLTPEAVSVELRPLMLWMANTTELLSFVQEKVLEMEKEADQEDPQLCNDLELCDEAMALLDEVIMCTFQQSVYYLTKTLYSTLPALLDSNPFTAGAELPGPGAELGAMPPGLRPTLGVFQAALELTSQCELHPDLVSQTFGYLFFFSNASLLNSLMERGQGRPFYQWSRAVQIRTNLDLVLDWLQGAGLGDIATEFFRKLSMAVNLLCVPRTSLLKASWSSLRTDHPTLTPAQLHHLLSHYQLGPGRGPPAAWDPPPAEREAVDTGDIFESFSSHPPLILPLGSSRLRLTGPVTDDALHRELRRLRRLLWDLEQQELPANYRHGPPVATSP

SEQ ID NO: 2 - protein, 961 aa, Mus musculus
MLSGERKEGGSPRFGKLHLPVGLWINSPRKQLAKLGRRWPSAASVKSSSSDTGSRSSEPLPPPPPPPHVELRRVGAVKAAGGASGSRAKRISQLFRGSGAGGAGGPGTPGGAQRWASEKKLPELAAGVAPEPPLPTRAAVPPGVLKIFASGLASGANYKSVLATERSTARELVAEALERYGLTGGRGAGDSGCVDAYALCDALGRPAVGVGGGEWRAEHLRVLADAERPLLVQDLWRARPGWARRFELRGREEARRLEQEAFGAADADGTNAPSWRTQKNRSRAASGGAALASPGPGSGSGTPTGSGGKERSENLSLRRSVSELSLQGRRRRQQERRQQALSMAPGAADAQMVPTDPGDFDQLTQCLIQAPSNRPYFLLLQGYQDAQDFVVYVMTREQHVFGRGGPSSSRGGSPAPYVDTFLNAPDILPRHCTVRAGPEPPAMVRPSRGAPVTHNGCLLLREAELHPGDLLGLGEHFLFMYKDPRSGGSGPARPSWLPARPGAAPPGPGWAFSCRLCGRGLQERGEALAAYLDGREPVLRFRPREEEALLGEIVRAAASGAGDLPPLGPATLLALCVQHSARELELGHLPRLLGRLARLIKEAVWEKIKEIGDRQPENHPEGVPEVPLTPEAVSVELRPLILWMANTTELLSFVQEKVLEMEKEADQEGLSSDPQLCNDLELCDEALALLDEVIMCTFQQSVYYLTKTLYSTLPALLDSNPFTAGAELPGPGAELEAMPPGLRPTLGVFQAALELTSQCELHPDLVSQTFGYLFFFSNASLLNSLMERGQGRPFYQWSRAVQIRTNLDLVLDWLQGAGLGDIATEFFRKLSIAVNLLCVPRTSLLKASWSSLRTDYPTLTPAQLHHLLSHYQLGPGRGPPPAWDPPPAERDAVDTGDIFESFSSHPPLILPLGSSRLRLTGPVTDDALHRELRRLRRLLWDLEQQELPANHRHGPPVASTP